(19) United States
(12) Patent Application Publication
(10) Pub. No.: US 2023/0125835 A1
(43) Pub. Date: Apr. 27, 2023

(54) SCOPE WITH CONTROLLABLE ENERGY TIP

(71) Applicant: Olympus Corporation, Tokyo (JP)

(72) Inventor: Joey Magno, Dudley, MA (US)

(21) Appl. No.: 17/938,544

(22) Filed: Oct. 6, 2022

Related U.S. Application Data
(60) Provisional application No. 63/363,077, filed on Apr. 15, 2022.
(60) Provisional application No. 63/262,877, filed on Oct. 21, 2021.

(51) Int. Cl. A61B 18/14; A61B 17/29

(52) U.S. Cl. CPC A61B 18/1445; A61B 17/2909; A61B 18/1482; A61B 2018/00494

(57) ABSTRACT

An endoscopic medical system can include an endoscopic end effector including three or more jaws. Each of the three or more jaws can respectively include a corresponding electromagnetic energy signal conductor. The end effector can also include two or more jaw linkages, an individual jaw linkage corresponding to a respective one of the jaws, such that at least two of the jaws are configured to move, independent of one another.

### CLAIM OF PRIORITY

This application claims priority to U.S. provisional Application Ser. No. 63/262,877, filed on Oct. 21, 2021, and Ser. No. 63/363,077, filed on Apr. 15, 2022, which are incorporated by reference herein in their entirety, and the benefit of priority of each of which is claimed herein.

### TECHNICAL FIELD

This document pertains generally, but not by way of limitation, to endoscopy and more particularly to operations for gastrointestinal bleeding.

### BACKGROUND

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In endoscopic treatment of gastrointestinal (GI) bleeding, a hemostasis technique can be used to treat a subject. Hemostasis techniques can be used, for instance, in the case of an emergency such as to treat a bleeding injury. Hemostasis techniques can be used to restrict blood flow such as to help enable a subject bodily response of coagulation or blood clotting. Here, the end effector can be coupled to an energy source and arranged to deliver energy to the tissue site of the bleeding injury, e.g., to help cauterize the site.

### OVERVIEW

The present disclosure relates to endoscopes for use in gastrointestinal procedures. Endoscopes described herein can help enable a skilled physician to precisely apply electromagnetic energy, such as radiofrequency (RF), to a target location of a patient to help coagulate tissue.

Example 1 can include or use subject matter such as an endoscopic medical system for delivering radiofrequency (RF) to a patient, the system comprising: an endoscopic end effector, comprising three or more jaws respectively including a corresponding electromagnetic energy signal conductor configured to be coupled to a radiofrequency or other electromagnetic energy signal generator; two or more jaw linkages, an individual jaw linkage corresponding to a respective one of the jaws, such that at least two of the jaws are configured to move, independent of one another, from a respective first jaw position to a respective second jaw position; and an actuator configured to actuate the at least two jaw linkages independent of one another to move corresponding jaws independent of one another from the respective first jaw position to a respective second jaw position.

Example 2 can include, or can optionally be combined with the subject matter of Example 1, wherein the actuator is further configured supply an RF signal to the at least two jaw linkages independent of one another and concurrent with moving an individual jaw to the first jaw position.

Example 3 can include, or can optionally be combined with the subject matter of Example 2, wherein the actuator is further configured to impede supply of an RF signal to the at least two jaw linkages independent of one another and concurrent with moving an individual jaw to the second jaw position.

Example 4 can include, or can optionally be combined with the subject matter of any of Examples 1-3, wherein at least one of the jaws includes an electrode configured to supply monopolar radiofrequency (MOP) to a target location of the patient.

Example 5 can include, or can optionally be combined with the subject matter of any of Examples 1-4, wherein the endoscopic end effector includes at least one anode and at least one cathode for supplying bipolar radiofrequency (BOP) to a target location of the patient.

Example 6 can include, or can optionally be combined with the subject matter of any of Examples 1-5, wherein the actuator is configured to move at the at least two jaw linkages independent of one another by retracting the corresponding jaw linkage.

Example 7 can include, or can optionally be combined with the subject matter of any of Examples 1-6, wherein the conductor includes a printed 3d printed metallic trace.

Example 8 can include, or can optionally be combined with the subject matter of any of Examples 1-7, wherein at least one of the jaws is constructed of a transparent or semi-transparent material.

Example 9 can include, or can optionally be combined with the subject matter of any of Examples 1-8, wherein at least two of the jaws are configured to grab a foreign body or tissue at the second jaw position.

Example 10 can include, or can optionally be combined with the subject matter of any of Examples 1-9, wherein the endoscopic medical system is attached to a distal tip of an endoscope and the actuator is disposed along a longitudinal axis of an insertion portion of the endoscope.

Example 11 can include or use subject matter such as an endoscopic medical system for delivering radiofrequency (RF) to a patient, the system comprising: an endoscopic end effector, comprising three or more jaws respectively including a corresponding electromagnetic energy signal conductor configured to be coupled to a radiofrequency or other electromagnetic energy signal generator; three or more jaw linkages, each individual jaw linkage corresponding to a respective one of the three or more jaws, such that each of the three or more jaws are configured to move, independent of one another, from a respective first jaw position to a respective second jaw position; and an actuator configured to actuate the three or more jaw linkages independent of one another to move corresponding jaws independent of one another from the respective first jaw position to a respective second jaw position.

Example 12 can include, or can optionally be combined with the subject matter of Example 11, wherein the actuator is further configured supply an RF signal to the three or more jaw linkages independent of one another and concurrent with moving an individual jaw to the first jaw position.

Example 13 can include, or can optionally be combined with the subject matter of Example 12, wherein the actuator is further configured to impede supply of an RF signal to the three or more jaw linkages independent of one another and concurrent with moving an individual jaw to the second jaw position.

Example 14 can include, or can optionally be combined with the subject matter of any of Examples 11-13, wherein at least one of the jaws includes an electrode configured to supply monopolar radiofrequency (MOP) to a target location of the patient.

Example 15 can include, or can optionally be combined with the subject matter of any of Examples 11-14, wherein the endoscopic end effector includes at least one anode and at least one cathode for supplying bipolar radiofrequency (BOP) to a target location of the patient.

Example 16 can include or use subject matter such as a method for performing endoscopy using radiofrequency (RF), the method comprising: providing or obtaining an endoscopic end effector including three or more jaws each corresponding with a respective jaw linkage; manipulating, via the jaw linkages, at least two of the three or more jaws independent of one another from a respective first jaw position to a respective second jaw position; and delivering, via at least one of the jaws, an electromagnetic energy signal to a patient tissue surface.

Example 17 can include, or can optionally be combined with the subject matter of Example 16, wherein delivering includes supplying an RF signal to the at least two jaw linkages independent of one another and concurrent with moving an individual jaw to the first jaw position.

Example 18 can include, or can optionally be combined with the subject matter of Example 17, further comprising impeding supply of an RF signal to the at least two jaw linkages independent of one another and concurrent with moving an individual jaw to the second jaw position.

Example 19 can include, or can optionally be combined with the subject matter of any of Examples 16-18, wherein delivering includes supplying monopolar radiofrequency (MOP) to a target location of the patient via an electrode included in or on at least one of the jaws.

Example 20 can include, or can optionally be combined with the subject matter of any of Examples 16-19, wherein delivering includes supplying bipolar radiofrequency (BOP) to a target location of the patient via at least one anode and at least one cathode included in or on the endoscopic end effector.

Example 21 can include or use subject matter such as at least one machine-readable medium including instructions that, when executed by processing circuitry, cause the processing circuitry to perform operations to implement of any of Examples 1-20.

Example 22 can include or use subject matter such as an apparatus comprising means to implement of any of Examples 1-20.

Example 23 can include or use subject matter such as a system to implement of any of Examples 1-20.

Example 24 can include or use subject matter such as a method to implement of any of Examples 1-20.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE FIGURES

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a perspective view depicting an example of a device for hemostasis.

FIG. 1B is a perspective view depicting an example of a device for hemostasis.

FIG. 1C is a diagrammatic view depicting an example of a device for hemostasis.

FIG. 1D is a diagrammatic view depicting an example of a device for hemostasis.

FIG. 2A is a cross sectional view of an end effector assembly attached at a distal end of an endoscope.

FIG. 2B is a cross sectional view of an end effector assembly attached at a distal end of an endoscope.

FIG. 2C depicts an end view of an end effector assembly.

FIG. 2D depicts an end view of an end effector assembly.

FIG. 2E depicts an end view of an end effector assembly.

FIG. 3A depicts a partial cross section of an example of an end effector assembly including a rotor.

FIG. 3B depicts an example of an actuator for use with an end effector assembly.

FIG. 3C depicts an example of an actuator for use with an end effector assembly.

FIG. 4A shows an end view including electrodes of an end effector assembly.

FIG. 4B shows a cross section including electrodes of an end effector assembly.

FIG. 5A shows an end view including electrodes of an end effector assembly.

FIG. 5B shows a cross section including electrodes of an end effector assembly.

FIG. 6 illustrates a flow diagram of an example of a method for performing endoscopy using radiofrequency.

### DETAILED DESCRIPTION

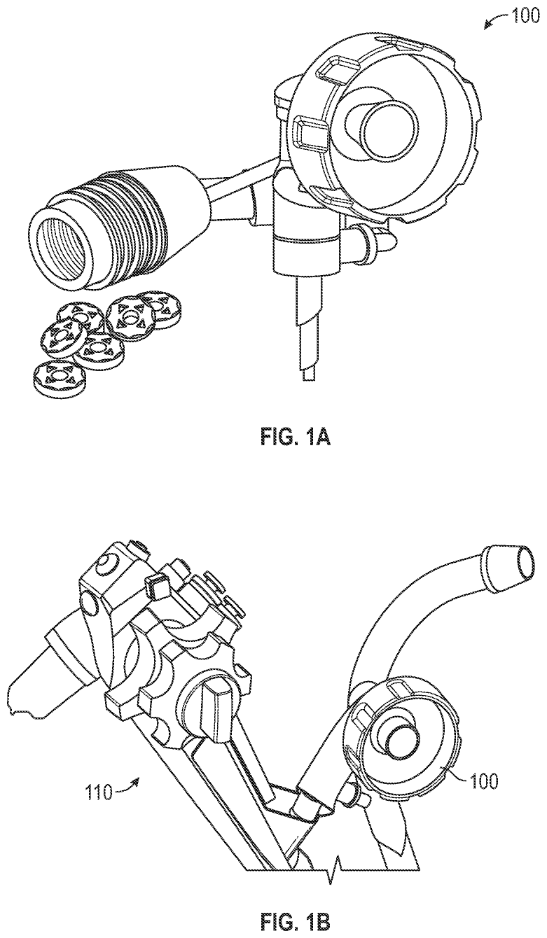

This disclosure describes apparatuses and methods for endoscopy and more particularly to operations such as hemostasis to treat gastrointestinal bleeding. FIG. 1A, FIG. 1B, FIG. 1C, and FIG. 1D depict an example of a device for hemostasis. In one approach to hemostasis of the gastrointestinal tract, an endoscopic variceal ligation (EVL) device, such as device 100, can be attached to the endoscope 110, and the target bleeding area is suctioned or pulled in to the EVL Device. In an example, as shown in FIG. 1C and FIG. 1D, a rubber band 120 can be used to constrict the area where bleeding occurs during suction using the endoscope 110. In another example, two separate endoscopes can be used for hemostasis to treat gastrointestinal bleeding: one for suction and one for EVL. A challenge with this approach is that it can take too much time to attach the EVL device to an endoscope during, e.g., a rapid treatment situation.

In yet another approach to hemostasis of the gastrointestinal tract, a local injection or spray of hemostatic agents can be included in treatment. Hemostatic methods such as water-absorbing and coagulating powders and gels that seal the bleeding area can be used. These hemostatic methods can be effective for bleeding that is difficult to treat (difficult access, too large hemostatic area, large amount of bleeding) or too costly to treat (multi-clipping, suturing, etc.). An example of this device can be a medical device composed of Absorbable Modified Polymer (AMP) particles and an applicator with a tube connecting to a clean external gas source. The particles can be delivered through a catheter inserted through the working channel of an endoscope which provides access to the site of the bleed. This approach can be used for hemostasis of certain types of non-variceal gastrointestinal bleeding. This approach, however, may not be preferred such as for patients who are sensitive to starch or starch-derived tissues or materials.

It can be challenging to adapt certain endoscopes to an emergency operation such as “emergency bleeds” in the gastric anatomy. Since endoscopes are not used frequently in an emergency room (ER) or intensive care unit (ICU) environment, sufficient facilities or personnel for cleaning reusable endoscopes are generally unavailable. Instead, single use (SU) endoscopes can be used in ER/ICU without the need to provide cleaning systems at the facility and to train the personnel in such cleaning procedures. A challenge with using some SU endoscopes is they often cannot handle emergency bleeding and therefore may not be a suitable replacement for reusable large channel scopes. The present inventors have realized, among other things, the need for a devices or methods to help enable efficient and effective hemostasis in a wide variety of treatment settings such as emergency treatment in an ER or ICU.

This disclosure describes apparatuses and methods for endoscopy and more particularly to operations for gastrointestinal medical procedures. Herein, endoscopes can relate to disposable/single-use endoscopes, bronchoscopes, duodenoscopes, laryngoscopes, fiberscopes, and other types of endoscopes. A similar SU endoscope such as for providing improved suction performance is described in U.S. Provisional patent application Ser. No. ______ entitled FLEXIBLE SCOPE WITH IMPROVED SUCTION, (Attorney Docket No. 5409.589PV2), filed on even date herewith, which is incorporated by reference herein in its entirety. The present disclosure describes an end effector including a plurality of portions. Herein, the plurality of portions can also be referred to as jaws, sections, or quadrants. Each of the plurality of portions can be independently energized, independently manipulated, or both. Each of the plurality of portions can be manipulated and energized simultaneously and independently from one another. For example, linkages can each be coupled to a respective portion and can be actuatable to manipulate their respective portion of the plurality of portions. The actuation of an individual linkage can also energize the respective portion connected to the linkage. The end effector assembly can be sized and shaped to be removably couplable to a distal end of an endoscope. The end effector assembly can also be integrated into the endoscope. Consequently, a single endoscope including the end effector assembly can be used with a dedicated working channel to remove foreign bodies and a monopolar or bipolar probe tip to coagulate an emergency bleed with its own dedicated working channel.

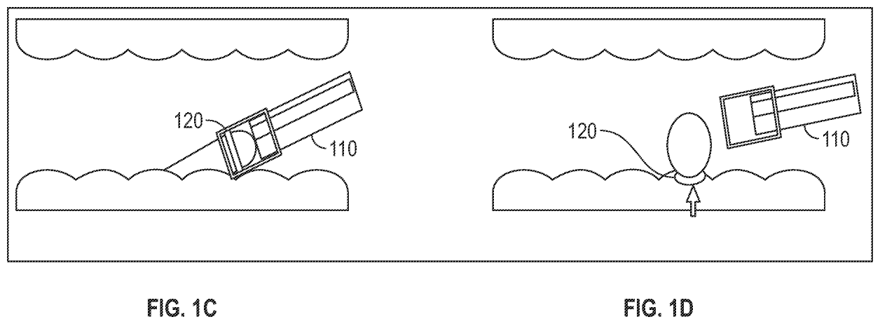

FIG. 2A and FIG. 2B depict an end effector assembly attached at a distal end of an endoscope. In an example, an end effector assembly 202 can be removably couplable to a distal end of an endoscope 210. The assembly 202 can be an attachment or extension to the endoscope 210 and can help impede a foreign object 204 from nearing or entering the endoscope at or near a plane 206 defining an active area at the distal end of the endoscope 210. This protection over the plane 206 can help prevent clogging of a working channel or a suction channel 212 of the endoscope 210 and can also help maintain a clear field of view around the plane 206. For example, the end effector assembly 202 can be formed of a transparent or semi-transparent material such as a transparent polymer or thermoplastic including polymethylmethacrylate, cellulose acetate butyrate, polycarbonate, glycol-modified polyethylene terephthalate, or combinations thereof. The distal extension/attachment can also include or use a viewing window or other viewing features to provide viewing through a non-transparent, semi-transparent, or transparent material. As shown in FIG. 2A, the foreign object may be too large to pass through the suction CH (channel) 212 of the endoscope 210. Here, the suction pressure can hold the foreign object at the tip of the end effector assembly 202.

The tip can have multiple portions 214, e.g., four quadrants 214 each having respective monopolar coils or traces that can be over-molded or printed on the external surfaces thereof. These multiple portions 214 can each be attached to a respective pull wire 216 such that operating the pull wires 216 can expand or collapse each respective portion 214. While described herein as pull wires 216, other types of linkages, e.g., rods, push wires, telescoping mechanisms, and the like can be alternatively or additionally used to manipulate corresponding multiple portions 214. For example, manipulating the pull wires 216 can move the portions 214 from a first position (depicted in FIG. 2A) to a second position (depicted in FIG. 2B). For example, the one or more of the pull wires 216 can be operated to manipulate one or more of the corresponding portions 214, to draw the portions 214 inward towards a central cylindrical axis 220 of the endoscope 210 (represented by point 220 in the end view of FIG. 2C, FIG. 2D, & FIG. 2E). All portions, A, B, C, and D form a dome-shape to coagulate a bleeding tissue site 218.

FIG. 2C, FIG. 2D, and FIG. 2E depict end views of an end effector assembly. Here, exemplary quadrants 214: A, B, C, and D are depicted. For example, the one or more pull wires 216 (as depicted in FIG. 2A & FIG. 2B) can be operated to manipulate a corresponding quadrant 214. Any combination of the portions 214 can be manipulated. For example, as in FIG. 2C, all four quadrants 214, A, B, C, and D, can be drawn in by their respective pull wires concurrently, simultaneously, or asynchronously to form the depicted orientation. As in FIG. 2D, two of four opposing quadrants, such as B and D, can be drawn in by their respective pull wires concurrently, simultaneously, or asynchronously to form the depicted orientation. As in FIG. 2E, two of four adjacent quadrants, such as A and B, can be drawn in by their respective pull wires concurrently, simultaneously, or asynchronously to form the depicted orientation. Other similar orientations can be formed with end effector assemblies having an amount other than four portions 214, such as three, five, or six portions 214. Also, other orientations can be formed having selected portions 214 drawn in, such as one single quadrant drawn in, three adjacent quadrants drawn in, or other permutations.

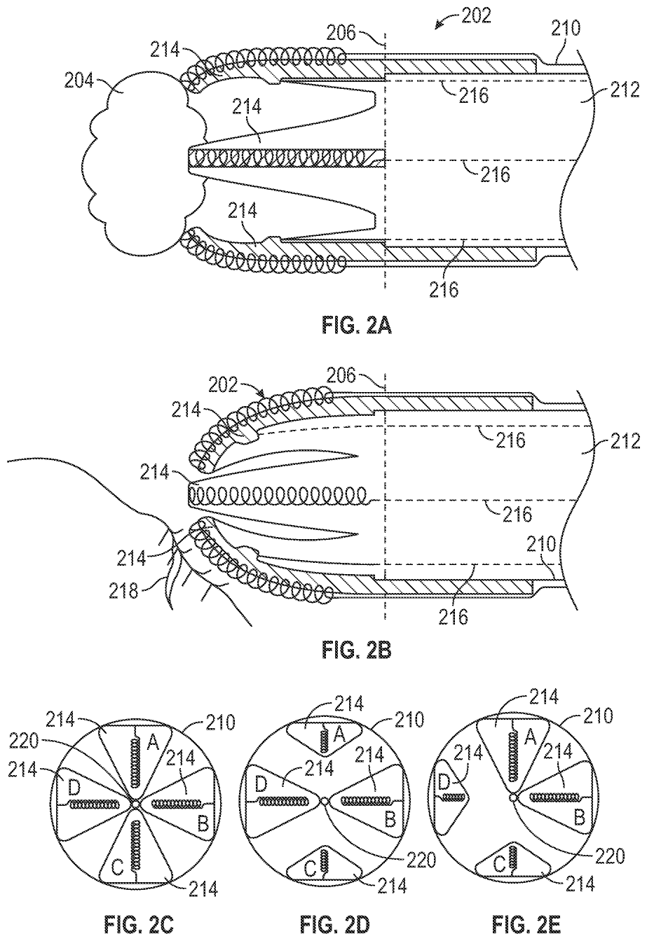

FIG. 3A depicts an example of an end effector assembly including a rotor. The drawing of the portions 214 inward towards the central cylindrical axis of the endoscope shows the ability to “grab” a foreign object. This grabbing feature can be used alternatively or additionally with suction, such as where suction alone would be insufficient to pull the foreign body close. Once “grabbed”, one or more rotors 222, e.g., including an auger or screw, embedded within the end effector assembly or the CH of the endoscope can grind, fragment, or break the foreign body down to smaller pieces to aid in extraction.

FIG. 3B and FIG. 3C depict an example of an actuator for use with an end effector assembly. For example, the actuator 224 can include a joystick which can be located at a proximal end of the endoscope at a handle or handpiece. The actuator 224 can also include mechanical, digital, or analog buttons arranged for similar ergonomic actuation to that of the joystick. For example, in the end-view depicted in FIG. 3C, the joystick of the actuator 224 can operate the linkages, e.g., the pull wires described above, by pushing the joystick in one of multiple directions a, b, c, and d to selectively draw inward towards the second position any of the respective corresponding portions, such as portions 214 A, B, C, and D as depicted in FIG. 2C-2E. In an example, pushing the joystick in an intermediate direction, such as a direction bisecting directions a and b, can concurrently or simultaneously draw inward to the second position two of the respective corresponding portions, such as portions 214 A and B as depicted in FIG. 2C-2E. Pushing the joystick in a certain direction, such as in towards the page in direction e, can concurrently or simultaneously draw all portions inward towards the second position. The magnitude of pressure applied to the actuator 224 can determine how far the selected portion(s) travel between the first position or the second position. For example, applying a threshold pressure to the actuator 224 can move the selected portion(s) to the second position. Also, applying less than the threshold pressure to the actuator 224 can move the selected portion(s) to an intermediate position between the first position and the second position. As such, each of the multiple portions can be independently or concurrently/simultaneously manipulated to move to one of several angular positions towards and away from the central cylindrical axis 220 of the endoscope 210 (represented by point 220 in the end view of FIG. 2C, FIG. 2D, & FIG. 2E). For example, the multiple portions can be manipulated in a range of motion between a definite number of digital positions. Also, the multiple portions can be manipulated in a range of motion between an indefinite number of graduating positions.

In an example, the actuator 224 can operate to supply an RF signal to the at least two portions 214 independent of one another and concurrently or simultaneously with moving an individual portion 214 towards the first position. The actuator 224 can include or use, e.g., an electrical switch or other electrical component that selectively conducts electricity for a specific jaw when a respective pull wire 216 is activated by the actuator 224 to move the individual portion 214 towards the first position. Also, the actuator 224 can operate to impede supply of an RF signal to the at least two portions 214 independent of one another and concurrent with moving an individual portion 214 towards the second position. Here, the switch or other electrical component can disconnect or block continuity of the RF signal such as to stop supply of the RF signal to the individual portion 214 when the respective pull wire 216 is activated by the actuator 224 to move the individual portion 214 towards the second position.

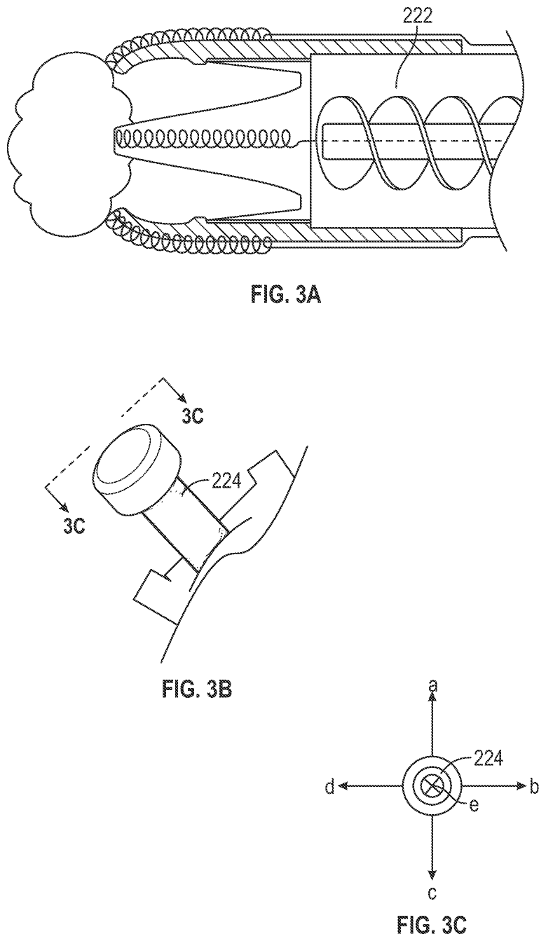

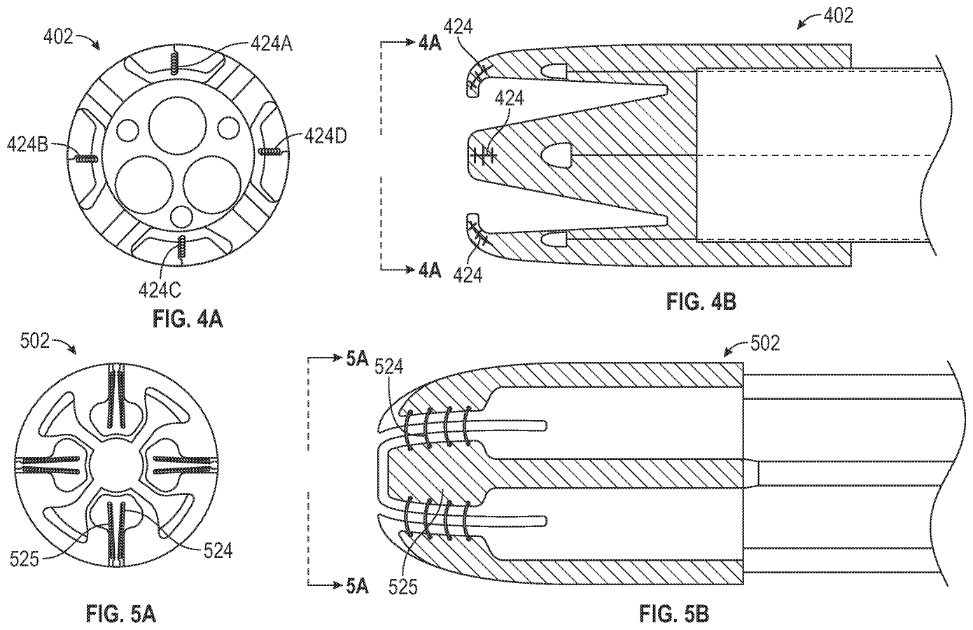

FIG. 4A, FIG. 4B, FIG. 5A, and FIG. 5B depict electrodes included in an end effector assembly. The endoscope depicted in FIG. 4A, FIG. 4B, FIG. 5A, and FIG. 5B can be substantially similar to the endoscopes described above. The components, structures, configurations, functions, etc. of the endoscope, such as the end effector assemblies 402 and 502 can therefore be the same as or substantially similar to that described in detail above, such as the end effector assembly 202.

An example of an end effector assembly 402 shown in FIG. 4A and FIG. 4B can include a plurality of electrodes 424, each electrode arranged on one of the portions. For example, the end effector assembly 402 can supply monopolar electromagnetic or RF energy to the tissue site from each of the plurality of electrodes 424A-D. A separate ground, such as a pad, trace, or line, can be applied to the patient. In another example, the end effector assembly 402 can supply bipolar electromagnetic or RF energy to the tissue site from the plurality of electrodes 424, and each electrode can correspond with an opposing electrode to supply the bipolar energy. For example, opposing electrodes 424A and 424C can form an anode-cathode pair, and bipolar electromagnetic energy can travel therebetween.

Another example of an end effector assembly 502 shown in FIG. 5A and FIG. 5B can include an electrode pair 524/525 arranged on an individual portion. For example, the end effector assembly 502 can supply bipolar electromagnetic or RF energy to the tissue site from the electrode pair 524/525, and each electrode (e.g., electrode 524) of the electrode pair 524/525 can correspond with an adjacent electrode (e.g., electrode 525) to supply the bipolar energy. For example, adjacent electrodes 524 and 525 can form an anode-cathode pair, and bipolar electromagnetic energy can travel therebetween.

Electrodes described herein can include overmolded coils. The electrodes can also include one or more embedded, impregnated, or lined electrically conductive traces. The electrodes can include a conductive material such as stainless steel, brass, silver, copper, or gold trace. The electrical trace can be printed or applied to one of the individual portions. For example, the trace can be laser printed using conductive ink, flexographically printed, silk screened, etched, or soldered to an individual portion.

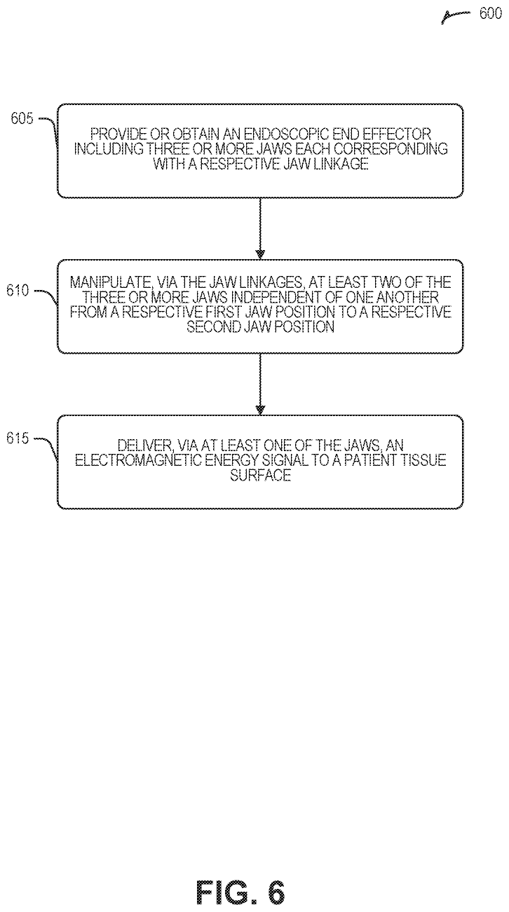

FIG. 6 illustrates a flow diagram of an example of a method 600 for performing endoscopy using radiofrequency, according to an example.

At operation 605, an endoscopic end effector can be provided or obtained, and the end effector can include or use three or more jaws each corresponding with a respective jaw linkage.

At operation 610, at least two of the three or more jaws can be manipulated, via the jaw linkages, independent of one another from a respective first jaw position to a respective second jaw position.

And, at operation 615, an electromagnetic energy signal can be delivered, via at least one of the jaws, to a patient tissue surface. Such delivery can include, e.g., supplying an RF signal to the at least two jaw linkages independent of one another and concurrent with moving an individual jaw to the first jaw position. Such delivery can also include supplying monopolar radiofrequency (MOP), bipolar radiofrequency (BOP), or both to a target location of the patient via an electrode included in or on at least one of the jaws. Also, supply of an RF signal can be impeded to or blocked from the at least two jaw linkages independent of one another and concurrent with moving an individual jaw to the second jaw position.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more Examples thereof), either with respect to a particular example (or one or more Examples thereof), or with respect to other examples (or one or more Examples thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more Examples thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations.

What is claimed is:

1. An endoscopic medical system for delivering radiofrequency (RF) to a patient, the system comprising:
an endoscopic end effector, comprising three or more jaws respectively including a corresponding electromagnetic energy signal conductor configured to be coupled to a radiofrequency or other electromagnetic energy signal generator;
two or more jaw linkages, an individual jaw linkage corresponding to a respective one of the jaws, such that at least two of the jaws are configured to move, independent of one another, from a respective first jaw position to a respective second jaw position; and
an actuator configured to actuate the at least two jaw linkages independent of one another to move corresponding jaws independent of one another from the respective first jaw position to the respective second jaw position.

2. The endoscopic medical system of claim 1, wherein the actuator is further configured to supply an RF signal to the at least two jaws independent of one another and concurrent with moving an individual jaw towards the first jaw position.

3. The endoscopic medical system of claim 2, wherein the actuator is further configured to impede supply of an RF signal to the at least two jaws independent of one another and concurrent with moving an individual jaw towards the second jaw position.

4. The endoscopic medical system of claim 1, wherein at least one of the jaws includes an electrode configured to supply monopolar radiofrequency (MOP) to a target location of the patient.

5. The endoscopic medical system of claim 1, wherein the endoscopic end effector includes at least one anode and at least one cathode for supplying bipolar radiofrequency (BOP) to a target location of the patient.

6. The endoscopic medical system of claim 1, wherein the actuator is configured to move at the at least two jaw linkages independent of one another by retracting the corresponding jaw linkage.

7. The endoscopic medical system of claim 1, wherein the conductor includes a printed metallic trace.

8. The endoscopic medical system of claim 1, wherein at least one of the jaws is constructed of a transparent or semi-transparent material.

9. The endoscopic medical system of claim 1, wherein at least two of the jaws are configured to grab a foreign body or tissue at the second jaw position.

10. The endoscopic medical system of claim 1, wherein the endoscopic medical system is attached to a distal tip of an endoscope and the actuator is disposed along a longitudinal axis of an insertion portion of the endoscope.

11. An endoscope attachment comprising:
a tubular member configured to be attached to a distal end of an endoscope;
three or more jaws respectively including a corresponding electromagnetic energy signal conductor configured to be coupled to a radiofrequency or other electromagnetic energy signal generator;
two or more jaw linkages, each individual jaw linkage corresponding to a respective one of the three or more jaws, such that each of the two or more jaws are configured to move, independent of one another, from a respective first jaw position to a respective second jaw position.

12. The endoscopic attachment of claim 11, wherein the three or more jaws are configured to receive an RF signal independent of one another and concurrent with moving an individual jaw towards the first jaw position.

13. The endoscopic attachment of claim 12, wherein the three or more jaws are configured to impede supply or not receive supply of an RF signal independent of one another and concurrent with moving an individual jaw towards the second jaw position.

14. The endoscopic attachment of claim 11, wherein at least one of the jaws includes an electrode configured to supply monopolar radiofrequency (MOP) to a target location of a patient.

15. The endoscopic attachment of claim 11, wherein the three or more jaws collectively include at least one anode and at least one cathode for supplying bipolar radiofrequency (BOP) to a target location of a patient.

16. A method for performing endoscopy using radiofrequency (RF), the method comprising:
providing or obtaining an endoscopic end effector including three or more jaws each corresponding with a respective jaw linkage;
manipulating, via the jaw linkages, at least two of the three or more jaws independent of one another from a respective first jaw position to a respective second jaw position; and
delivering, via at least one of the jaws, an electromagnetic energy signal to a patient tissue surface.

17. The method of claim 16, wherein delivering includes supplying an RF signal to the at least two jaw linkages independent of one another and concurrent with moving an individual jaw to the first jaw position.

18. The method of claim 17, further comprising impeding supply of an RF signal to the at least two jaw linkages independent of one another and concurrent with moving an individual jaw to the second jaw position.

19. The method of claim 16, wherein delivering includes supplying monopolar radiofrequency (MOP) to a target location of the patient via an electrode included in or on at least one of the jaws.

20. The method of claim 16, wherein delivering includes supplying bipolar radiofrequency (BOP) to a target location of the patient via at least one anode and at least one cathode included in or on the endoscopic end effector.
